(19) United States
(12) Plant Patent Application Publication
(10) Pub. No.: US 2003/0093845 P1
(43) Pub. Date: May 15, 2003

(54) Hybrid tea rose plant named 'Meizoele'

(76) Inventor: Alain A. Meilland, Antibes (FR)

Correspondence Address: BURNS, DOANE, SWECKER & MATHIS, L.L.P., P.O. Box 1404, Alexandria, VA, 22313-1404, US

(21) Appl. No.: 10/288,426

(22) Filed: Nov. 6, 2002

Related U.S. Application Data
(63) Continuation of application No. 09/499,673, filed on Feb. 8, 2000, now abandoned.

(51) Int. Cl. A01H 5/00

(52) U.S. Cl. PLT/130

(57) ABSTRACT

A new and distinct variety of Hybrid Tea rose plant is provided which abundantly forms attractive double bicolored blossoms that are yellow bordered with red in coloration. The plant exhibits a vigorous growth habit, semi-glossy dark green foliage, and very good disease resistance. The dark green foliage contrasts nicely with the lighter colored blossoms. The new variety is particularly well suited for growing as attractive ornamentation in the landscape such as in parks and gardens.

### SUMMARY OF THE INVENTION

The new variety of Hybrid Tea rose plant was created by artificial pollination wherein two parents were crossed which previously had been studied in the hope that they would contribute the desired characteristics. The female parent (i.e., the seed parent) of the new variety was the ‘Meinarval’ variety (non-patented in the United States). The male parent (i.e., the pollen parent) was the ‘Lapdal’ variety (non-patented in the United States). The parentage of the new variety can be summarized as follows:

### ‘Meinarval’×‘Lapdal’.

The seeds resulting from the above pollination were sown and small plants were obtained which were physically and biologically different from each other. Selective study resulted in the identification of a single plant of the new variety.

It was found that the new variety of Hybrid Tea rose plant of the present invention possesses the following combination of characteristics:

(a) exhibits a vigorous growth habit,

(b) abundantly forms attractive double bicolored blossoms that are yellow bordered with red in coloration,

(c) forms attractive dark green semi-glossy foliage that contrasts well with the blossoms, and

(d) is particularly well suited for growing as attractive ornamentation in the landscape.

The disease resistance of the new variety is very good.

The new variety well meets the needs of the horticultural industry and can be grown to advantage in parks and gardens.

The new variety has been found to undergo asexual propagation in France by a number of routes, including budding, grafting, and cuttage. Asexual propagation by the above-mentioned techniques in France has shown that the characteristics of the new variety are stable and are strictly transmissible by such asexual propagation from one generation to another.

The new variety has been named the ‘Meizoele’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during October while budded on Rosa froebelli understock and growing in outdoors at Le Cannet des Maures, Var, France. Dimensions in centimeters are indicated at the bottom of the photograph.

FIG. 1 -illustrates a specimen of a young shoot;

FIG. 2 -illustrates a specimen of a floral bud before the opening of the sepals;

FIG. 3 -illustrates a specimen of a floral bud at the opening of the sepals;

FIG. 4 -illustrates a specimen of a floral bud at the opening of the petals;

FIG. 5 -illustrates a specimen of a flower in the course of opening;

FIG. 6 -illustrates a specimen of an open flower—plan view—obverse;

FIG. 7 -illustrates a specimen of an open flower—plan view—reverse;

FIG. 8 -illustrates a specimen of a fully open flower—plan view—obverse;

FIG. 9 -illustrates a specimen of a fully open flower—plan view—reverse;

FIG. 10 -illustrates a specimen of a floral receptacle showing the arrangement of the stamens and pistils;

FIG. 11 -illustrates a specimen of a floral receptacle showing the arrangement of the pistils (stamens removed);

FIG. 12 -illustrates a specimen of a flowering stem;

FIG. 13 -illustrates a specimen of a main branch;

FIG. 14 -illustrates a specimen of a leaf with three leaflets—plan view - upper surface; and

FIG. 15 -illustrates a specimen of a leaf with five leaflets—plan view - under surface.

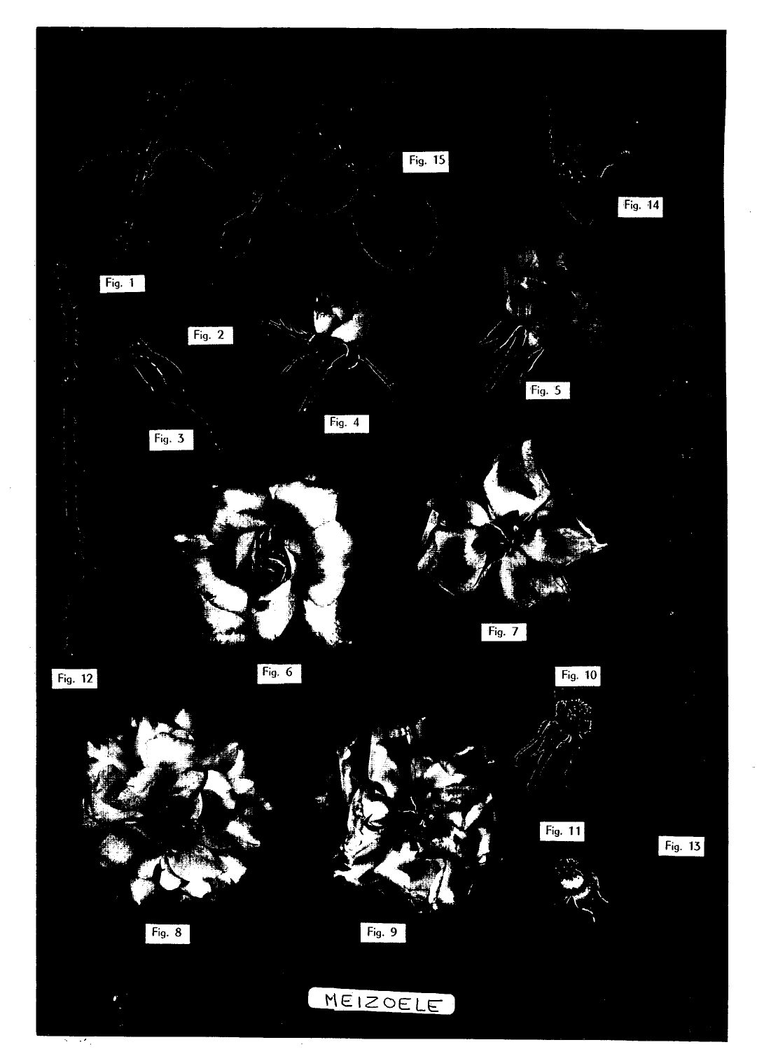

### DETAILED DESCRIPTION

The chart used in the identification of the colors is that of The Royal Horticultural Society (R.H.S. Colour Chart). The description is based on the observation of two year-old plants during October while budded on Rosa froebelli understock and growing outdoors at Le Cannet des Maures, Var, France. The coloration in common terms precedes reference to the chart in some instances. Such terminology is to be accorded its ordinary dictionary significance.

CLASS: Hybrid Tea.

PLANT: height—approximately 80 to 90 cm. at the end of the second growing season.

habit.—semi-erect.

BRANCHES: color—young stems: Yellow-Green Group 146A. adult wood: lighter green, Yellow-Green Group 146C.

thorns.—size: large (as illustrated). quantity: medium (as illustrated). color: pinkish to reddish on young stems and Havana brown, Greyed-Brown Group 165B, on adult wood.

LEAVES: stipules—adnate, pectinate, and rather broad.

petioles.—upper surface: Yellow-Green Group 148A and non-glandular. under surface: commonly free of prickles.

leaflets.—number: 3, 5 (most often), and 7. shape: symmetrical at the tip and obtuse at the base. serration: single and regular (as illustrated). texture: consistent. general appearance: dark green and semi-glossy foliage. color (young foliage): upper surface: Green Group 138A. under surface: lighter green, Green Group 138B. color (adult foliage):

upper surface: dark green, Green Group 139A. under surface: Yellow-Green Group 147A.

INFLORESCENCE

number of flowers.—usually one flower per stem.

peduncle.—rigid, medium in thickness, Yellow-Green Group 147C, and the length is approximately 6 cm. on average.

sepals.—upper surface: tomentose and greenish, Yellow-Green Group 148C, with some reddish coloration. under surface: Yellow-Green Group 146B, and commonly with some extensions (as illustrated).

buds.—shape: globular. size: large. length: approximately 3 cm. on average. color upon opening: upper surface: near Yellow-Orange Group 14A to 14C suffused with Red Group 46A to 46C. under surface: near Yellow-Orange Group 14B to 14C suffused with Red Group 46A to 46C.

flower.—shape: cup-shaped. diameter: approximately 11 to 12 cm. on average. color (when opening begins): upper surface: near Yellow-Orange Group 14A to 14C suffused with Red Group 46A to 46C. under surface: near Yellow-Orange Group 14B to 14C suffused with Red Group 46A to 46C. color (when blooming): upper surface: near Yellow Group 12A to 12D bordered with Red Group 47C. under surface: near Yellow Group 12B to 12D bordered with Red Group 47C. color (at end of opening): upper surface: near Yellow Group 12A to 12D bordered with Red Group 47C. under surface: near Yellow Group 12B to 12D bordered with Red Group 47C. lasting quality: very long. petal number: commonly approximately 35 to 40 on average. petal shape: wedge-shaped and oblong. petal drop: good, the petals commonly detach cleanly. stamen number: approximately 109 on average. anthers: near Yellow-Orange Group 15C in coloration. filaments: near Yellow-Orange Group 15C in coloration. pistils: approximately 95 on average. stigmas: near Yellow Group 8D in coloration. styles: near Red Group 40D in coloration. receptacle: near Yellow-Green Group 146B in coloration, and funnel-shaped in longitudinal section.

DEVELOPMENT

vegetation.—vigorous.

blooming.—very abundant.

resistance to.—

diseases.—very good.

resistance to.—

frost.—very good.

fructification.—poor.

I claim:

1. A new and distinct variety of Hybrid Tea rose plant characterized by the following combination of characteristics:
(a) exhibits a vigorous growth habit,
(b) abundantly forms attractive double bicolored blossoms that are yellow bordered with red in coloration,
(c) forms attractive dark green semi-glossy foliage that contrasts well with the blossoms, and
(d) is particularly well suited for growing as attractive ornamentation in the landscape; substantially as herein shown and described.
